(19) United States
(12) Patent Application Publication
(10) Pub. No.: US 2003/0148949 A1
(43) Pub. Date: Aug. 7, 2003

(54) Methods and compositions for treating dermal lesions

(76) Inventor: Daniel K. Podolsky, Wellesley, MA (US)

Correspondence Address: CLARK & ELBING LLP, 101 FEDERAL STREET, BOSTON, MA, 02110, US

(21) Appl. No.: 10/266,069

(22) Filed: Oct. 7, 2002

Related U.S. Application Data
(60) Provisional application No. 60/327,673, filed on Oct. 5, 2001.

(51) Int. Cl. A61K 38/17

(52) U.S. Cl. 514/12

(57) ABSTRACT

This invention features methods of treating and preventing damage to the epidermis and dermis by local administration of trefoil peptides. The trefoil peptide can be administered either alone or in combination with other therapeutics including antimicrobial agents, anti-inflammatory agents or, analgesics.

### CROSS REFERENCE TO RELATED APPLICATIONS

This application claims benefit of the filing date of U.S. Provisional Application No. 60/327,673, filed Oct. 5, 2001, hereby incorporated by reference.

### FIELD OF INVENTION

This invention relates to methods and compositions for treating and preventing lesions of the skin in a mammal that can result from traumatic, infective, physiologic, or pathologic causes.

### BACKGROUND OF THE INVENTION

Full thickness wounds of the skin can result from various causes, the most common of which are traumatic or surgical lesions. Partial thickness wounds are commonly caused by abrasions, burns, pressure injuries, or other minor trauma. Skin epithelial destruction can also be a consequence of cancer chemotherapy or radiotherapy of the skin. Skin lesions can further result from a hypersensitive reaction to a therapeutic agent either administered topically or systemically. In addition to those drugs which can cause direct damage, certain drugs including many antibiotics, can induce skin photosensitivity, which may in turn lead to epithelial lesions. Alternatively, wounds and ulcers can also result from vascular insufficiency; from chronic diabetes, which is often characterized by vascular diseases; as well as from pressure necrosis and bedsores.

Typically, routine wound care is directed at reducing infection, ensuring an adequate arterial supply and venous drainage, and in cases of moderate or severe injury, ensuring the close approximation of the epithelial surfaces by mechanical methods (e.g., sutures or wound clips). In this regard, secondary infections by pathogenic microorganisms are important to consider, particularly in light of the protective barrier function of the skin. These conditions, when severe, are risk factors for chronic debilitating local infections and septicemias as microorganisms may use the compromised epithelium as a portal of entry into the body. Secondary infections may be further exacerbated in immunocompromised patients, such as those undergoing cancer treatment (chemotherapy or radiotherapy).

In addition to the functional restoration of the dermal barrier, wound care is further concerned with the cosmetic outcome and the reduction of scar tissue. As such, the rapid restoration of a normal epithelial layer has the potential to reduce the amount of scar formation and secondary complications of the wound, particularly infections.

### SUMMARY OF THE INVENTION

This invention features the treatment and prevention of lesions of the skin of a mammal by administration to the lesion, or regions of the skin where a lesion is to be prevented, therapeutically effective amounts of a trefoil peptide, or a biologically active fragment thereof. Treatment or prevention of lesions according to the invention can speed healing, reduce pain, delay or prevent occurrence of the lesion, and inhibit expansion, secondary infection, or other complications of the lesion. Preferably, the mammal is a human. In particularly useful embodiments, the trefoil peptide is spasmolytic peptide (SP), pS2, Intestinal Trefoil Peptide (ITF), ITF₁₅₋₇₃, ITF₂₁₋₇₃, ITF₁₋₇₂, ITF₁₅₋₇₂, or ITF₂₁₋₇₂, and is present in a pharmaceutical composition containing a pharmaceutically acceptable carrier. Other useful trefoil peptides include polypeptides that are substantially identical to SP, pS2, ITF, ITF₁₅₋₇₃, ITF₂₁₋₇₃, ITF₁₅₋₇₂, or ITF₂₁₋₇₂. Preferably, the trefoil peptide is ITF or a biologically active ITF fragment, which may be administered as a monomer, a dimer, or another multimeric form.

The methods and compositions of this invention are particularly useful for treating lesions of the skin caused by an inflammatory or allergic reaction such as eczema, psoriasis, or contact dermatitis; pressure ulcers, acne, lesions caused by physical trauma or surgical intervention (e.g., local biopsy or cut), lesions caused by chemical, thermal or radiation burns, or lesions caused by anti-neoplastic therapy (e.g., chemotherapy or radiation therapy). Additionally, lesions of the skin that result from microbial infections, whether bacterial, viral (e.g., herpes or papilloma virus) or fungal, are also amenable to treatment. More specifically, administration of trefoil peptides is also useful for treating lesions or promoting epithelial growth and maturity in preterm infants whose epidermis is in an immature state.

In the methods and compositions, a second therapeutic agent can be included. Desirable second therapeutic agents include anti-inflammatory agents (e.g., rofecoxib or celecoxib), antibacterial agents (e.g., benzoyl peroxide, povidone iodine, azelaic acid, retinoids, clindamycin, erythromycin, penicillins, cephalosporins, tetracyclines, and aminoglycosides), antifungal agents (e.g., nystatin, amphotericin B, benzoic acid, undecylenic alkanolamide, ciclopirox olamine, polyenes, imidazole, allylamine, and thiocarbamate), antiviral agents (e.g., acyclovir), topical analgesics (e.g., lidocaine and benzocaine), systemic analgesics (e.g., opiates, fentenyl, and NSAIDS), steroids (e.g., triamcinolone, glucocorticoid, budesonide, fluocinolone, betamethasone, diflucortolone, fluticasone, mometasone, prednisone, methylprednisolone, betamethasone, dexamethasone, triamcinolone, and hydrocortisone), and ultraviolet blocking agents. Sedatives, such as the benzodiazepines (e.g., diazepam), may also be administered systemically in severe cases of shock associated with dermal trauma. Trefoil peptides when administered for the treatment of psoriasis may include topical agents (e.g., anthralin, retinoids, vitamin D analogues, and glucocorticoids) or systemic agents (e.g., methotrexate and cyclosporine). The second therapeutic agent may be administered within (either before or after administration of the trefoil peptide) 14 days, 7 days, 1 day, 12 hours, 1 hour, or simultaneously with the trefoil peptide.

The second therapeutic agent can be present in the same or different pharmaceutical compositions as the trefoil peptide. When the second therapeutic agent is present in a different pharmaceutical composition, different routes of administration may be used. For example, the second therapeutic may be administered orally, or by intravenous, intramuscular, or subcutaneous injection. Thus, the second therapeutic need not be administered topically.

Of course, pharmaceutical compositions may contain two, three, or more trefoil peptides or biologically active fragments. Alternatively, topical administration of the trefoil peptide may be supplemented by oral administration of the same or a different trefoil peptide.

The compositions of this invention can also be used prophylactically, prior to therapies or conditions that will damage the dermis or epidermis. For example, the compositions can be applied to an area of the skin prior to cancer therapy in order to mitigate the loss of epidermal integrity. Compositions containing a trefoil peptide can also be applied to an area of the skin prior to sun exposure or prior to a surgical intervention.

Suitable pharmaceutical compositions include at least one trefoil peptide and a pharmaceutically acceptable carrier. Treatment using trefoil peptidecontaining compositions of this invention is typically self-administered. However, trefoil peptide therapy may be administered by a medical professional or other health care provider. For example, a trefoil peptide-containing gel, cream, solution, suspension, ointment, spray, bioerodable polymer, or hydrogel (non-bioerodable polymer) may be applied to lesions caused by the removal of a malignant lesion immediately after a surgical or laser removal procedure. In other useful embodiments, a mucoadhesive, an osmotic agent, or viscosity-enhancing agent is present. Alternatively, the trefoil peptide can be formulated for topical application as a concentrated paste, suspension, a cream, or an ointment to be applied directly to the lesion. Alternatively, the trefoil peptide can be formulated as a topical patch to provide sustained delivery of the peptide. This patch may or may not be adhesive, and may or may not be occlusive. An occlusive patch may increase the permeability of trefoil peptide through a partially denuded epithelium. Other occlusive excipients (e.g., hydrophobic polymers) and also penetration enhancers (fatty acids, alcohols, benzoates, glycols, or pyrrolidones) may also be used to enhance trefoil penetration. Furthermore, the trefoil peptide can be formulated to irrigate a wound prior to suturing or during a surgical procedure. The trefoil peptide may therefore be formulated in an irrigation solution such as saline or Ringer's solution. Alternatively, trefoil peptides can be impregnated in suture material (gut, silks, collagens, glycolic acid polymers or nylon) or wound dressings (gauze pads, occlusive dressings, semi-occlusive dressings, alginates, hydrocolloids and adhesive films).

Mammalian trefoil peptides were discovered in 1982. One of the mammalian trefoil peptides, human intestinal trefoil factor (ITF; TFF3), has been characterized extensively, and is described in U.S. Pat. Nos. 6,063,755, and 6,221,840, hereby incorporated by reference. The other two known human trefoil peptides are spasmolytic polypeptide (SP; TFF2) and pS2 (TFF1). Trefoil peptides, described extensively in the literature (e.g., Sands et al., Annu. Rev. Physiol. 58: 253-273, 1996), are expressed in the gastrointestinal tract and have a three-loop structure formed by intrachain disulfide bonds between conserved cysteine residues. These peptides protect the intestinal tract from injury and can be used to treat intestinal tract disorders such as peptic ulcers and inflammatory bowel disease. Homologs of these human peptides have been found in a number of non-human animal species. All members of this protein family, both human and non-human, are referred to herein as trefoil peptides. Human ITF will be referred to most extensively in this application; however, the activity of human ITF is common to each of the mammalian trefoil peptides.

“Trefoil peptide,” as used herein, includes all mammalian homologs of human spasmolytic polypeptide (SP; also known as TFF2), human pS2 (also known as TFF1) and human intestinal trefoil factor (ITF; also known as TFF3), and biologically active fragments thereof. Homologs of the trefoil peptides have, preferably, 70% amino acid identity to the human sequence, more preferably 85% identity, most preferably 95%, or even 99% sequence identity. The length of comparison sequences will generally be at least about 10 amino acid residues, usually at least 20 amino acid residues, more usually at least 30 amino acid residues, typically at least 45 amino acid residues, and preferably more than 60 amino acid residues. Alternatively, trefoil peptides are polypeptides encoded by a polynucleotide that hybridizes with high stringency to the human ITF, pS2, or SP cDNAs provided in SEQ ID NOs: 4, 5, and 6, respectively, or the human ITF, pS2, or SP genes provided in SEQ ID NOs: 7, 8, and 9, respectively.

The term “fragment” is meant to include polypeptides that are truncations or deletions of SP, pS2 and ITF. Preferably, the fragments are biologically active and have 70% amino acid identity to the corresponding regions of the human polypeptide sequence. More preferably, the fragments are 85% identical, most preferably 95%, or even 99% identical to the human polypeptide sequence to which they correspond. The length of comparison sequences will generally be at least about 10 amino acid residues, usually at least 20 amino acid residues, more usually at least 30 amino acid residues, typically at least 45 amino acid residues, and preferably more than 60 amino acid residues.

Preferable fragments contain four cysteine residues in any positions which correspond to the cysteines at positions 25, 35, 45, 50, 51, 62, or 71, of human ITF (FIG. 1), or positions 31, 41, 51, 56, 57, 68, and 82 of human pS2 (FIG. 2). More preferably, fragments contain five cysteine residues at these positions. Most preferably, six, or even all seven cysteines are present.

Fragments of SP are meant to include truncations or deletions and preferably have 70% sequence identity to the corresponding human SP polypeptide sequence (FIG. 3). More preferably, the fragments are 85% identical, most preferably 95%, or even 99% identical to the human polypeptide sequence. Preferably, active fragments contain at least four cysteine residues, which correspond to positions 6, 8, 19, 29, 34, 35, 46, 58, 68, 78, 83, 84, 95, and 104 in the human SP polypeptide. More preferably, fragments contain six cysteines, which correspond to these positions. Even more preferable are fragments that contain eight cysteines. Most preferable are fragments that contain cysteines at ten, twelve, or even, all fourteen positions.

It is recognized in the art that one function of the identified cysteine residues is to impart the characteristic three-loop (trefoil) structure to the protein. Accordingly, preferred fragments of ITF and pS2 have a least one loop structure, more preferably, the fragments have two loop structures, and most preferably, they have three loop structures. It is equally well recognized that the native SP polypeptide has a six loop confirmation. Preferable fragments contain at least two of these loop structures, more preferably, four loop structures are conserved, and most preferably, five, or even all six loop structures are present.

By “co-formulated” is meant any single pharmaceutical composition, which contains two or more therapeutic or biologically active agents.

By “pharmaceutical preparation” or “pharmaceutical composition” is meant any composition, which contains at least one therapeutically or biologically active agent and is suitable for administration to a patient. For the purposes of this invention, pharmaceutical compositions suitable for delivering a therapeutic to the skin include, but are not limited to aqueous solutions, creams, gels, suspensions, sprays, bioerodable polymer, hydrogel (non-bioerodable gel polymer), patches, irrigation solution, pastes, lotions, ointments, foams, wound dressings, and sutures. Any of these formulations can be prepared by well-known and accepted methods of art. See, for example, Remingtion: The Science and Practice of Pharmacy, 19^{th} edition, (ed. A R Gennaro), Mack Publishing Co., Easton, Pa., 1995.

By “topical administration” is meant the application of a therapeutically effective amount of pharmaceutical composition to the external and/or exposed surface of the skin, to access the dermis and/or epidermis.

By “therapeutically effective amount” is meant an amount sufficient to provide medical benefit. When administering trefoil peptides to a human patient according to the methods described herein, an effective amount will vary with the size of the lesion area being treated; however, a therapeutically effective amount is usually about 1-2500 mg of trefoil peptide per dose. Preferably, the patient receives at least 10 mg, 100 mg, 500 mg, 750 mg, 1000 mg, 1500 mg, or 2000 mg of trefoil peptide in each dose. Larger amounts may be required for large lesions such as those caused by extensive thermal burns. Dosing is typically performed 1-5 times each day.

By “biologically active,” when referring to a trefoil peptide, fragment, or homolog is meant any polypeptide that exhibits an activity common to its related, naturally occurring family member, and that the activity is common to the family of naturally occurring trefoil peptides. An example of a biological activity common to the family of trefoil peptides is the ability to restitute the gastrointestinal mucosa (Taupin et al, Proc. Natl. Acad. Sci. USA. 97(2): 799-804).

By “isolated DNA” is meant DNA that is free of the genes, which in the naturally occurring genome of the organism from which the given DNA is derived flank the DNA. Thus, the term “isolated DNA” encompasses, for example, cDNA, cloned genomic DNA, and synthetic DNA.

By “treating” is meant administering a pharmaceutical composition for prophylactic and/or therapeutic purposes. The active ingredients of the pharmaceutical composition can treat the primary indication (e.g., epithelial lesion) or secondary symptoms (e.g., concomitant infection, pain, or inflammation).

By “burn” is meant any injury to the dermis, epidermis, or underlying tissue that results from exposure to heat, acids, caustics, chemicals, electricity, or radiation (e.g., ultraviolet radiation), marked by varying degrees of skin destruction and hyperemia often with the formation of watery blisters and in severe cases by charring of the tissues, and classified according to the extent and degree of the injury. There are three classifications of burns. A first-degree burn is superficial, involving only the top layer of the skin (epidermis). First-degree burns are characterized by dry, red skin, and typically heal within 5-6 days without permanent scarring. A second-degree burn is a partial thickness burn, involving the epidermis and the dermis. These burns will blister and weep, and in the absence of therapy, usually require 3-4 weeks or longer to heal. Scarring may occur. The most severe, third-degree burn, destroys all skin layers, and some of the underlying tissue. Complications of shock and infection make a third-degree burn potentially life threatening.

By “wound dressing” is meant any occlusive or semi-occlusive covering that overlay a lesion or injury site. In addition to providing contact of the skin with a trefoil peptide, preferable dressings maintain a moist environment at the lesion site, remove excess exudates, have thermal insulation properties, allow gaseous exchange, are impermeable to microorganisms, and/or allow trauma-free removal. The choice of dressing will be influenced, for example, by clinical indications such as the type of wound, wound position, presence of debris or infection, level of exudate, patient comfort, and cost efficiency.

By “antimicrobial agent” is meant any compound that alters the growth of bacteria or fungi cells, or viruses whereby growth is prevented, stabilized, or inhibited, or wherein the microbes are killed. In other words, the antimicrobial agents can be microbiocidal or microbiostatic.

By “antineoplastic therapy” is meant any treatment regimen used to treat cancer. Typical antineoplastic therapies include chemotherapy and radiation therapy.

By “ultraviolet blocking agent” is meant any treatment regimen used to block ultraviolet radiation. Typical ultraviolet radiation blockers are formulated as creams or pastes to be applied before sun exposure.

By “substantially identical” is meant a polypeptide or nucleic acid exhibiting at least 75%, but preferably 85%, more preferably 90%, most preferably 95%, or 99% identity to a reference amino acid or nucleic acid sequence. For polypeptides, the length of comparison sequences will generally be at least 20 amino acids, preferably at least 30 amino acids, more preferably at least 40 amino acids, and most preferably 50 amino acids. For nucleic acids, the length of comparison sequences will generally be at least 60 nucleotides, preferably at least 90 nucleotides, and more preferably at least 120 nucleotides.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65 C., or a buffer containing 48% formamide, 4.8× SSC, 0.2 M Tris-Cl, pH 7.6, 1× Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well known by those skilled in the art of molecular biology. See, e.g., F. Ausubel et al., Current Protocols in Molecular Biology, John Wiley & Sons, New York, N.Y., 1998, hereby incorporated by reference. Other features and advantages of the invention will be apparent from the following detailed description, and from the claims.

### BRIEF DESCRIPTION OF THE DRAWINGS

FIG. 1 is an amino acid sequence of a human intestinal trefoil factor (ITF; Accession No. BAA95531).

FIG. 2 is an amino acid sequence of a human pS2 protein (Accession No. NP_{—}003216).

FIG. 3 is an amino acid sequence of human spasmolytic polypeptide (SP; Accession No. 1909187A).

FIG. 4 is a cDNA sequence encoding a human intestinal trefoil factor.

FIG. 5 is a cDNA sequence encoding a human pS2 protein.

FIG. 6 is a cDNA sequence encoding a human spasmolytic polypeptide.

FIG. 7 is the nucleotide sequence of a gene encoding human intestinal trefoil factor (locus 10280533:52117-55412).

FIG. 8 is the nucleotide sequence of a gene encoding human pS2 protein (locus 10280533:16511-21132).

FIG. 9 is the nucleotide sequence of a gene encoding human spasmolytic polypeptide (locus 10280533:957-5208).

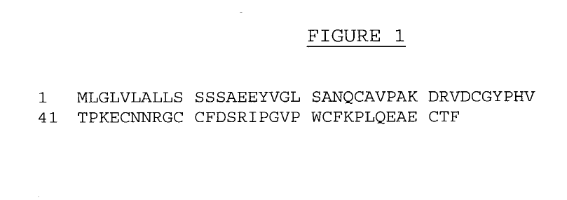

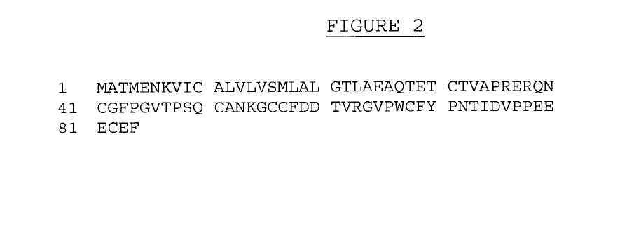

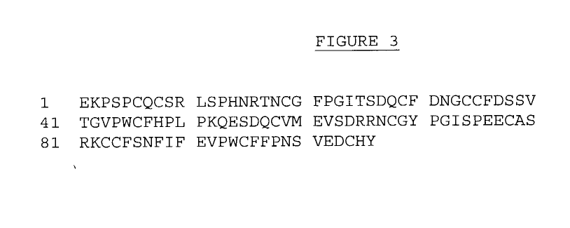

### DETAILED DESCRIPTION

The invention provides methods and compositions useful for the treatment of a wide range of lesions to the dermis and epidermis. Lesions may occur on any part of the human skin, including for example the scalp, groin and uro-genital area, face, trunk, arms hands, legs, soles of the feet or between the toes. Lesions of the dermis and epidermis amenable to treatment according to the present invention can be induced by physical trauma (e.g., cuts, abrasions, and surgical intervention), chemical and thermal burns (e.g., sunburn), vascular compromise (e.g., resulting from diabetes), infective or inflammatory processes (e.g., eczema, psoriasis, contact dermatitis, herpetic lesion, and acne), microbial infection (e.g., viral, bacterial, and fungal), or antineoplastic therapy (e.g., radiotherapy). In the case of burn patients, particularly those in which skin damage inflicted by the burns is severe and extends to a large proportion of the body, epidermal loss may deteriorate rapidly due to both heat and water transpiration. Although mechanical systems such as hydrogels have been used to alleviate this problem, rapid restoration of an adequate barrier is imperative for the recovery of such patients.

Preterm infants also suffer from impaired skin barrier function owing to the immature state of the epidermis and the relative absence of the stratum corneum in such infants. Although, the degree of severity is largely dependent on gestational age, compromise to the skin barrier can ultimately cause significant complications. In this respect, the increased permeability of the skin to water leads to significant heat dissipation and also provides a nesting site for foreign substances, including for example allergens, microorganisms, and toxins. Overall, these lesions are treated by local application of trefoil peptides either alone or in combination with another therapeutic agent.

Pharmaceutical Formulations: Ointments, Pastes, Creams, Gels, Irrigation Solutions, and Tissue Adhesives

Lesions of the epithelium of the skin, such as those resulting from trauma or inflammation, are amenable to trefoil peptide therapy delivered as an ointment, paste, or gel. The viscous nature of these types of preparations allows for direct application to the wound site. Optionally, the wound site can be covered with a dressing to retain the trefoil peptide-containing composition, protect the lesion and/or absorb exudate. As discussed further below, these preparations are particularly useful to restore epithelial integrity following traumatic surgical procedures (e.g., skin biopsies and incisions). Such viscous formulations may also have a local barrier effect thereby reducing irritation and pain. In addition, trefoil peptides can also be present in any of the known irrigation solutions (e.g. 0.9% saline or Ringer's solution) used for surgery purposes.

Mucoadhesives

A mucoadhesive excipient can be added to any of the previously described pharmaceutical compositions. The mucoadhesive formulations coat the lesioned area, resulting in retention of the trefoil peptide at the lesion site, providing protection, inhibiting irritation, and accelerating healing of inflamed or damaged tissue. Mucoadhesive formulations suitable for use in these pharmaceutical preparations are well known in the art (e.g., U.S. Pat. No. 5,458,879). Particularly useful mucoadhesives are hydrogels composed of about 0.05-20% of a water-soluble polymer such as, for example, poly (ethylene oxide), poly (ethylene glycol), poly (vinyl alcohol), poly (vinyl pyrrolidine), poly (acrylic acid), poly (hydroxy ethyl methacrylate), hydroxyethyl ethyl cellulose, hydroxy ethyl cellulose, chitosan, and mixtures thereof. These polymeric formulations can also contain a dispersant such as sodium carboxymethyl cellulose (0.5-5.0%).

Other preferred mucoadhesive excipients for liquid compositions are ones that allow the composition to be administered as a flowable liquid but will cause the composition to gel on the skin, thereby providing a bioadhesive effect which acts to hold the therapeutic agents at the lesion site for an extended period of time. The anionic polysaccharides pectin and gellan are examples of materials which when formulated into a suitable composition will gel on the skin, owing to the presence of cations in the mucosal fluids. The liquid compositions containing pectin or gellan will typically consist of 0.01-20% w/v of the pectin or gellan in water or an aqueous buffer system.

Other useful compositions, which promote mucoadhesion and prolonged therapeutic retention in the dermis and epidermis, are colloidal dispersions containing 2-50% colloidal particles such as silica or titanium dioxide. Such formulations form as a flowable liquid with low viscosity; however, the particles interact with glycoprotein, especially mucin, transforming the liquid into a viscous gel, providing effective mucoadhesion (e.g., U.S. Pat. Nos. 5,993,846 and 6,319,513).

Bioadhesives and biocrodable polymers are useful as an alternative method of wound closure, or as a drug delivery vehicle. Bioadhesives are a particularly useful alternative to sutures, for wound closure in geriatric populations, where the skin is particularly friable. Any of the well-known bioadhesives or polymers is suitable for use with the trefoil peptides of this invention (e.g. U.S. Pat. Nos. 5,990,194, 6,159,498, and 6,284,235). The trefoil peptides are incorporated into the adhesive or polymer by any method suitable for incorporating any other therapeutic agent into these products. The particular method will depend on the chemical composition of the product and the manufacturing process.

Medical Materials

Suture materials, sterile wound dressings (occlusive and semi-occlusive, e.g., gauze pads), topical patches, adhesive films, and tissue adhesives can be impregnated with the trefoil peptides of the present invention and used at an incision site to promote dermal and epidermal healing. Any of the suture materials, wound dressings, topical patches, adhesive films, and tissue adhesives may also contain ITF-consisting biocrodable polymers and alginates.

These formulations can be made according to known and conventional methods for preparing such formulations. For example, sutures made from monofilaments can be impregnated by loading the polymer solution with a trefoil peptide, prior to extrusion. Suture material can also be impregnated by repeated soaking/drying cycles using a trefoil peptide-containing solution. The number of cycles depends on the concentration of trefoil peptide in the soaking solution and the final amount of peptide to be contained in the suture. Soaking is a particularly effective impregnation method for braided suture materials because the trefoil peptide is retained by the surface contours.

Sterile dressings and gauzes for wounds and burns, impregnated with a trefoil peptide, can also be prepared by standard methods. Typically, the trefoil peptide will be present in a viscous gel (e.g., hydrogel), separated from the dermal lesion by a permeable fabric that does not adhere to the wound.

Therapeutic Agents

Trefoil Peptides

The therapeutic trefoil peptide(s) are typically mammalian trefoil peptides or fragments thereof. Preferably, human trefoil peptides or fragments are used; however, trefoil peptides from other species including rat, mouse, and non-human primate, may be used. Typically, the trefoil peptide is intestinal trefoil factor (ITF); however, spasmolytic polypeptide (SP), or pS2 are also useful.

The trefoil peptides or fragments are administered at 1-5000 mg per dose, preferably 5-2500 mg per dose, or more preferably 10-1500 mg per dose, depending on the nature and condition of the lesion being treated, the anticipated frequency and duration of therapy, and the type of pharmaceutical composition used to deliver the trefoil peptide. The trefoil peptides are typically administered 1-5 times per day.

Therapeutic Fragments of Intestinal Trefoil Factor (ITF)

Particular ITF fragments retain biological activity and may be substituted in any method or composition in which ITF is used. Methods and compositions containing ITF, in which these ITF fragments may be substituted, are described, for example, in U.S. Pat. Nos. 6,063,755 and 6,221,840, and U.S. patent application Nos. 10/131,363, filed Apr. 24, 2002, 60/317,657, filed Sep. 6, 2001, 60/327,673, filed Oct. 5, 2002, 60/333,836, filed Nov. 28, 2001, and 60/367,574, filed Mar. 26, 2002 (hereby incorporated by reference).

Particularly useful ITF fragments that retain biological activity include the polypeptide corresponding to amino acid residues 15-73 of SEQ ID NO:1 (ITF₁₅₋₇₃) and amino acid residues 21-73 of SEQ ID NO:1 (ITF₂₁₋₇₃). Other useful ITF fragments are formed following cleavage of the C-terminal phenylalanine residue (i.e., ITF₁₋₇₂, ITF₁₅₋₇₂, and ITF₂₁₋₇₂).

The biologically active ITF fragments of this invention can be produced using any appropriate method. For example, cDNA encoding the desired ITF fragment can be used with any method known in the art for producing recombinant proteins. Exemplary methods are provided herein. ITF fragments, particularly ITF₂₁₋₇₃, can be produced using a Pichia yeast expression system (see, for example, U.S. Pat. Nos. 4,882,279 and 5,122,465) transformed with a cDNA encoding long ITF species, such as the full length ITF (e.g., SEQ ID NO:4) or ITF₁₅₋₇₃, when the fermentation culture is maintained at pH˜5.0.

Anti-Inflammatory Agents

Any suitable anti-inflammatory agent can be formulated with the trefoil peptide and employed using the method of this invention. Suitable anti-inflammatory agents include, but are not limited to non-steroidal anti-inflammatory drugs (e.g., ibuprofen and tacrolimus), cyclooxygenase-2-specific inhibitors such as rofecoxib (Vioxx®) and celecoxib (Celebrex®). Anti-inflammatory concentrations known to be effective following administration can be used. For example, ibuprofen may be present in the composition at concentrations sufficient to deliver between 25-800 mg per day to the lesion.

Antimicrobial Agents

Any of the many known antimicrobial agents can be used in the compositions of the invention at concentrations generally used for these agents. Antimicrobial agents include antibacterials, antifungals, and antivirals.

Although the most widely used antibacterial agents used for the skin are benzoyl peroxide, povidone iodine, azelaic acid, retinoids, clindamycin and erythromycin, other examples of antibacterial agents (antibiotics) include the penicillins (e.g., penicillin G, ampicillin, methicillin, oxacillin, and amoxicillin), the cephalosporins (e.g., cefadroxil, ceforanid, cefotaxime, and ceftriaxone), the tetracyclines (e.g., doxycycline, minocycline, and tetracycline), the aminoglycosides (e.g., amikacin, gentamycin, kanamycin, neomycin, streptomycin, and tobramycin), the macrolides (e.g., azithromycin, clarithromycin, and erythromycin), the fluoroquinolones (e.g., ciprofloxacin, lomefloxacin, and norfloxacin), and other antibiotics including chloramphenicol, clindamycin, cycloserine, isoniazid, rifampin, and vancomycin.

Antiviral agents are substances capable of destroying or suppressing the replication of viruses. Examples of anti-viral agents include 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir.

Antifungal agents include both fungicidal and fungistatic agents such as, for example, benzoic acid, undecylenic alkanolamide, ciclopirox olamine, polyenes, imidazoles, allylamine, thicarbamates, amphotericin B, butylparaben, clindamycin, econaxole, fluconazole, flucytosine, griseofulvin, nystatin, and ketoconazole.

Antimicrobial concentrations known to be effective following topical administration can be used. For example, tetracycline may be present in the composition at concentrations that are known to provide between 100-1000 mg per day to the lesion following topical application.

Analgesics and Anesthetics

Any of the commonly used topical analgesics can be used in the compositions of the invention. The analgesic is present in an amount such that there is provided to the skin lesion a concentration of between one-half and five percent concentration for lidocaine (e.g., 5-50 mg/ml in 20-40 ml per dose of liquid). Examples of other useful anesthetics include procaine, lidocaine, tetracaine, dibucaine, benzocaine, p-buthylaminobenzoic acid 2-(diethylamino) ethyl ester HCl, mepivacaine, piperocaine, and dyclonine.

Other analgesics may be administered systemically, including opioids such as, for example, morphine, codeine, hydrocodone, and oxycodone. Any of these analgesics may also be co-formulated with other compounds having analgesic or anti-inflammatory properties, such as acetaminophen, aspirin, and ibuprofen.

Steroids

Steroids may be used to treat lesions of the skin and formulated to be used in the compositions of the present invention. Typically, topical steroid agents employed include but are not limited to fluocinolone, triamcinolone, betamethasone, diflucortolone, fluticasone, hydrocortisone, mometasone, methylprednisolone, and clobetasol. In extreme cases of skin irritation, systemic steroid agents such as prednisone, prednisolone, meythylprednisolone, betamethasone, dexamethasone, triamcinolone, and hydrocortisone, may also be administered.

Dosages

All of the therapeutic agents employed in the topical compositions of the present invention, including the trefoil peptide component, can be used in the dose ranges currently known and used for these agents. The following are illustrative examples of dose ranges for the active ingredients of the compositions of the invention. Different concentrations of either the trefoil peptide or the other agents may be employed depending on the clinical condition of the patient, the goal of therapy (treatment or prophylaxis), and anticipated duration or severity of the damage for which the agent is being given. Additional considerations in dose selection include: disease etiology, patient age (pediatric, adult, geriatric), general health and comorbidity.

Production of Trefoil Peptides

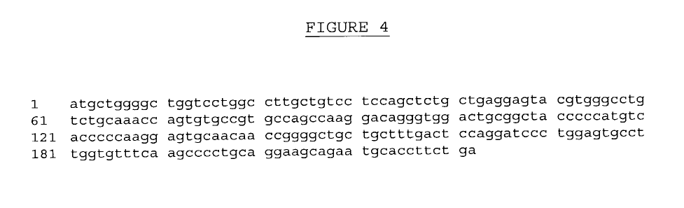

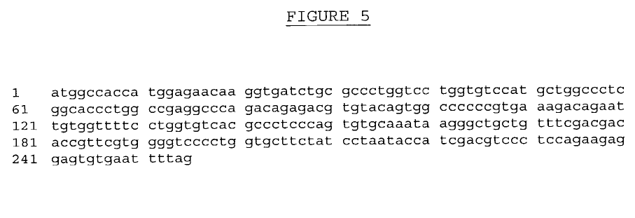

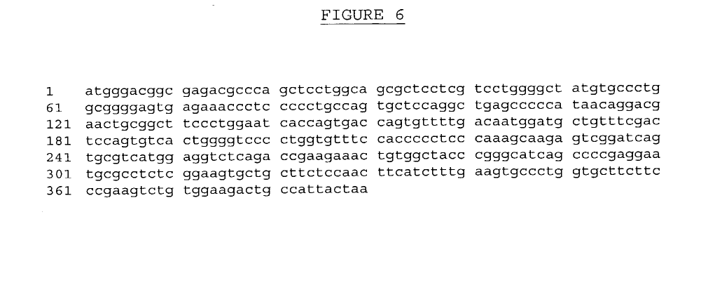

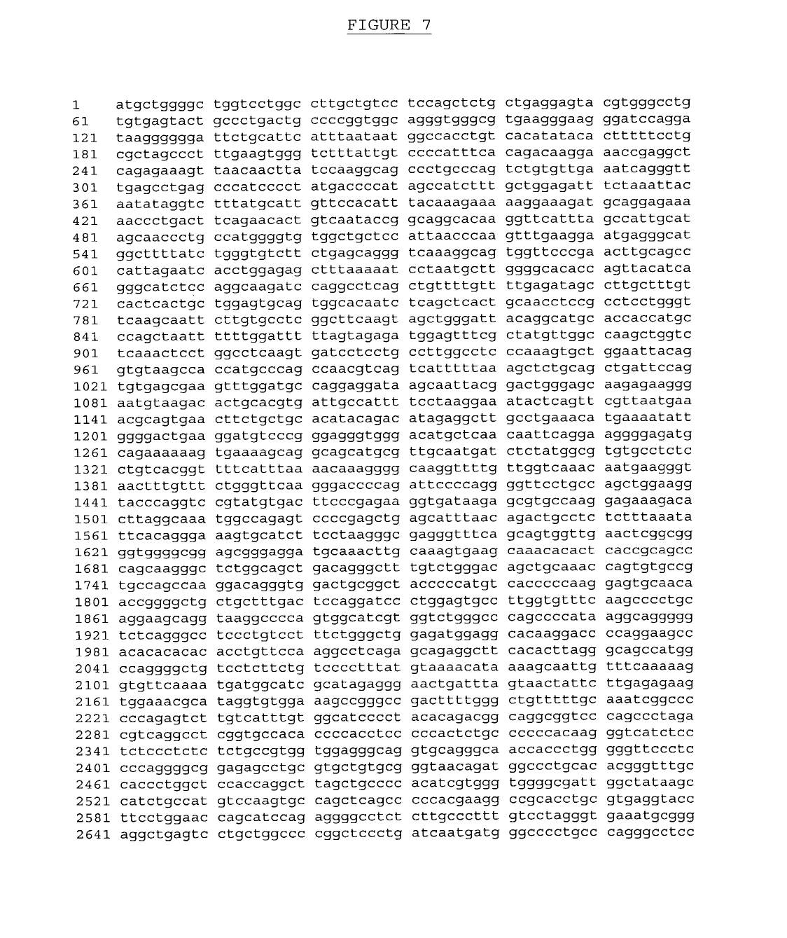

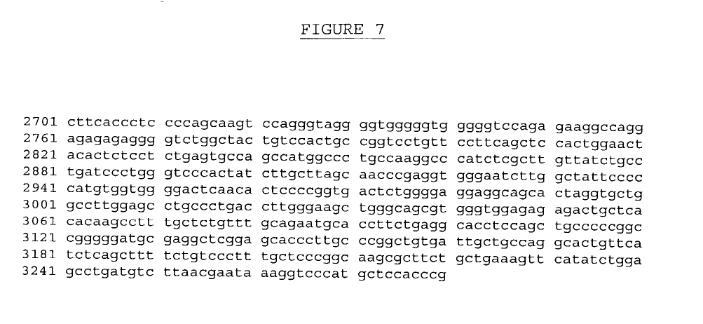

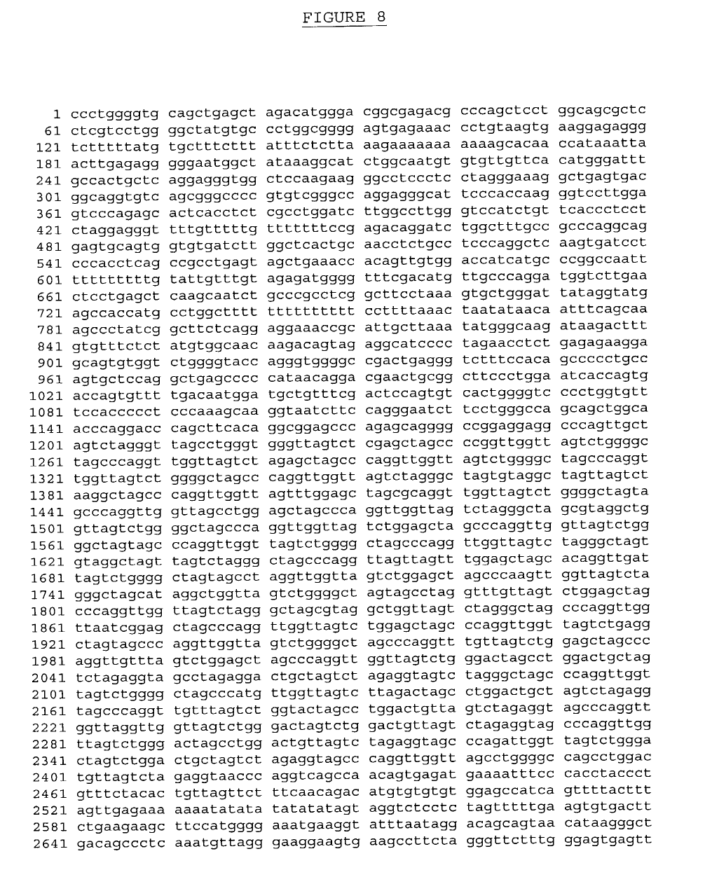

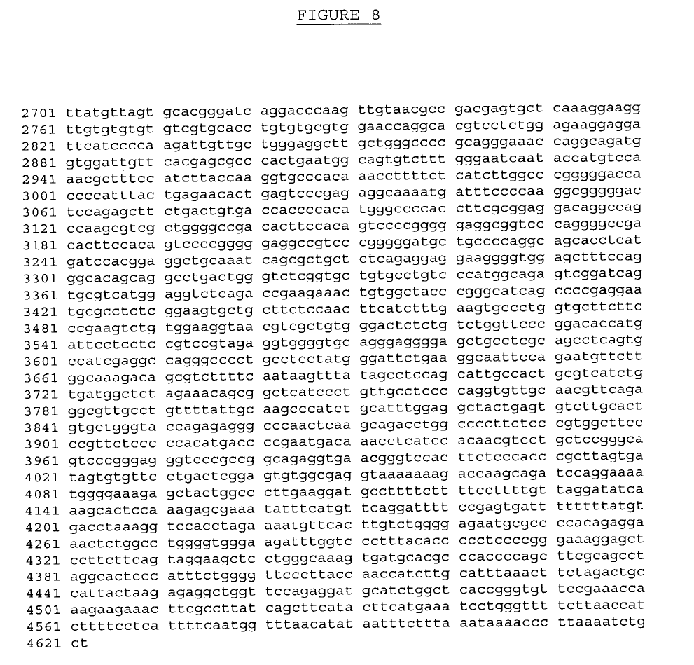

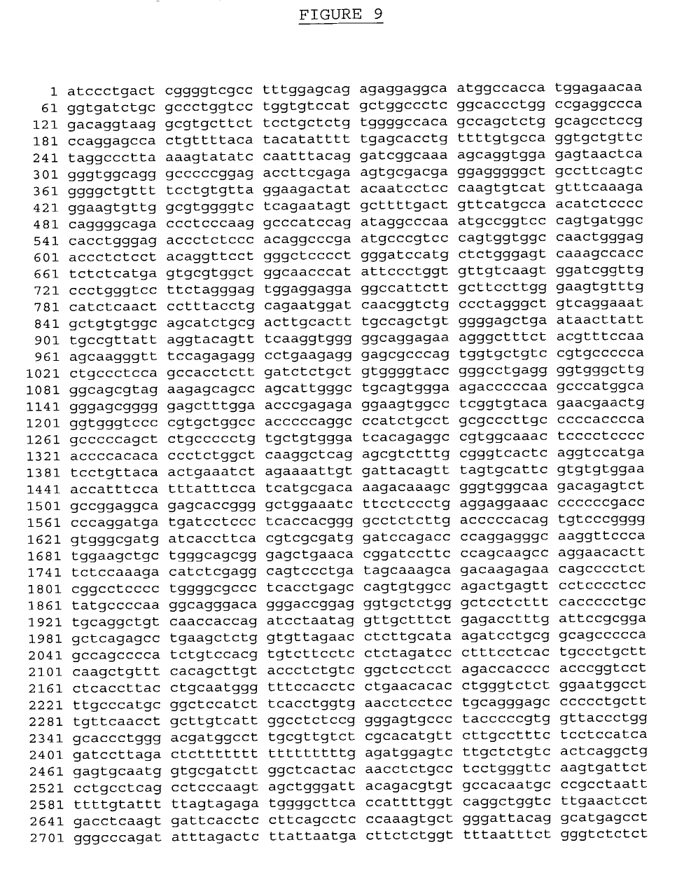

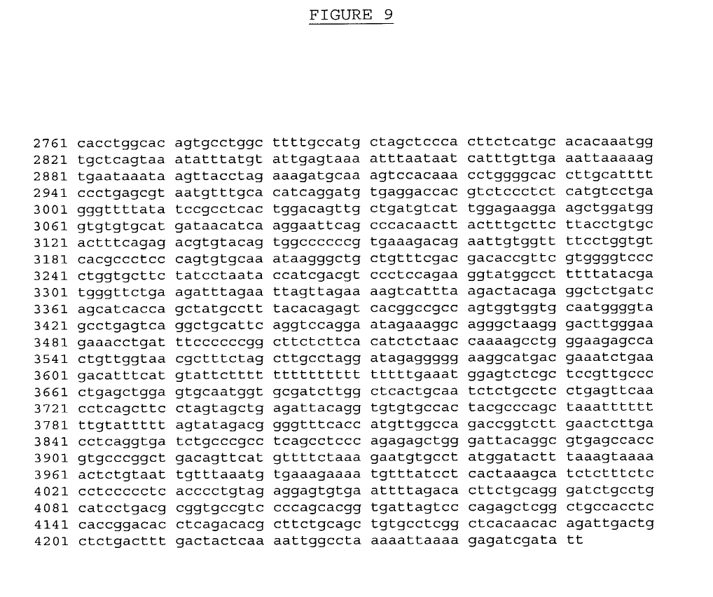

Trefoil peptides and fragments can be produced by any method known in the art for expression of recombinant proteins. Nucleic acids that encode trefoil peptides (e.g., human intestinal trefoil factor (FIGS. 4 and 7), human pS2 (FIGS. 5 and 8), and human spasmolytic polypeptide (FIGS. 6 and 9) or fragments thereof may be introduced into various cell types or cell-free systems for expression thereby allowing large-scale production, purification, and patient therapy.

Eukaryotic and prokaryotic trefoil peptide expression systems may be generated in which a trefoil peptide gene sequence is introduced into a plasmid or other vector, which is then used to transform living cells. Constructs in which the trefoil peptide cDNA contains the entire open reading frame inserted in the correct orientation into an expression plasmid may be used for protein expression. Prokaryotic and eukaryotic expression systems allow for the expression and recovery of trefoil peptide fusion proteins in which the trefoil peptide is covalently linked to a tag molecule, which facilitates identification and/or purification. An enzymatic or chemical cleavage site can be engineered between the trefoil peptide and the tag molecule so that the tag can be removed following purification.

Typical expression vectors contain promoters that direct the synthesis of large amounts of mRNA corresponding to the inserted trefoil peptide nucleic acid in the plasmid-bearing cells. They may also include a eukaryotic or prokaryotic origin of replication sequence allowing for their autonomous replication within the host organism, sequences that encode genetic traits that allow vector-containing cells to be selected for in the presence of otherwise toxic drugs, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

Expression of foreign sequences in bacteria, such as Escherichia coli, requires the insertion of a trefoil peptide nucleic acid sequence into a bacterial expression vector. Such plasmid vectors contain several elements required for the propagation of the plasmid in bacteria, and for expression of the DNA inserted into the plasmid. Propagation of only plasmid-bearing bacteria is achieved by introducing, into the plasmid, selectable marker-encoding sequences that allow plasmid-bearing bacteria to grow in the presence of otherwise toxic drugs. The plasmid also contains a transcriptional promoter capable of producing large amounts of mRNA from the cloned gene. Such promoters may be (but are not necessarily) inducible promoters that initiate transcription upon induction. The plasmid also preferably contains a polylinker to simplify insertion of the gene in the correct orientation within the vector. Mammalian cells can also be used to express a trefoil peptide. Stable or transient cell line clones can be made using trefoil peptide expression vectors to produce the trefoil peptides in a soluble (truncated and tagged) form. Appropriate cell lines include, for example, COS, HEK293T, CHO, or NIH cell lines.

Once the appropriate expression vectors are constructed, they are introduced into an appropriate host cell by transformation techniques, such as, but not limited to, calcium phosphate transfection, DEAE-dextran transfection, electroporation, microinjection, protoplast fusion, or liposome-mediated transfection. The host cells that are transfected with the vectors of this invention may include (but are not limited to) E. coli or other bacteria, yeast, fungi, insect cells (using, for example, baculoviral vectors for expression in SF9 insect cells), or cells derived from mice, humans, or other animals. In vitro expression of trefoil peptides, fusions, or polypeptide fragments encoded by cloned DNA may also be used. Those skilled in the art of molecular biology will understand that a wide variety of expression systems and purification systems may be used to produce recombinant trefoil peptides and fragments thereof. Some of these systems are described, for example, in Ausubel et al. (Current Protocols in Molecular Biology, John Wiley & Sons, New York, N.Y. 2000, hereby incorporated by reference).

Transgenic plants, plant cells and algae are also particularly useful for generating recombinant trefoil peptides for use in the methods and compositions of the invention. For example, transgenic tobacco plants or cultured transgenic tobacco plant cells expressing a trefoil peptide can be created using techniques known in the art (see, for example, U.S. Pat. Nos. 5,202,422 and 6,140,075). Transgenic algae expression systems can also be used to produce recombinant trefoil peptides (see, for example, Chen et al., Curr. Genet. 39:365-370, 2001).

Once a recombinant protein is expressed, it can be isolated from cell lysates using protein purification techniques such as affinity chromatography. Once isolated, the recombinant protein can, if desired, be purified further by e.g., high performance liquid chromatography (HPLC; e.g., see Fisher, Laboratory Techniques In Biochemistry And Molecular Biology, Work and Burdon, Eds., Elsevier, 1980).

Polypeptides of the invention, particularly trefoil peptide fragments can also be produced by chemical synthesis using, for example, Merrifield solid phase synthesis, solution phase synthesis, or a combination of both (see, for example, the methods described in Solid Phase Peptide Synthesis, 2nd ed., 1984, The Pierce Chemical Co., Rockford, Ill.). Optionally, peptide fragments are then be condensed by standard peptide assembly chemistry.

The following examples are intended to illustrate the principle of the present invention and circumstances when trefoil peptide therapy is indicated. The following examples are not intended to be limiting.

### EXAMPLE 1

### ITF Therapy Following A Surgical Intervention

To speed healing of a surgical incision and reduce scar formation, the surgical patient is administered ITF-containing preparations using a variety of modalities. Just prior to surgery, an ITF-containing gel is applied to the skin at the site of impending incision. Following the surgical procedure, the incision is irrigated with sterile saline, or another irrigation solution, containing 25 mg/ml ITF. The incision is closed using ITF-impregnated suture silk and the incision site is treated with a paste or gel preparation containing 5 mg/ml ITF. The ITF-containing paste or gel is reapplied during each dressing change. Preferably, the paste or gel is reapplied for at least three days, more preferably for five days, most preferably for seven days, or even ten days, or until the incision is completely healed, and the sutures are removed or absorbed by the body.

### EXAMPLE 2

### Burn Treatment

The ultimate goal of burn-wound management is closure and healing of the wound. In this respect, scarring is a common result of second-and third-degree burns. Given that scars are formed when the proliferative and migratory capacity of fibroblasts exceed that of the epithelial cells, therapies that promote epithelial restitution will reduce scar formation. In addition, it is known that topical or systemic administration of an antimicrobial can dramatically decrease the bacterial burden of burn wounds and reduce the incidence of burn-wound infection.

Three widely used topical antimicrobial agents, namely silver sulfadiazine cream, mafenide acetate cream, and silver nitrate can be admixed, alone or in combination, with ITF to treat burn wounds. Burn victims are therefore treated with a paste or gel containing 1% silver sulfadiazine, mafenide acetate cream and/or silver nitrate, along with a topical analgesic, and 10 mg/ml ITF. Topical treatment is re-applied every 12 hours for the duration of therapy. In addition to topical application of the ITF-containing ointment, the burn site may be wrapped in an ITF-impregnated bandage.

Normally, analgesic and antibiotic therapy will be terminated once satisfactory capillary development and epithelialization has occurred. In order to minimize scarring, ITF therapy is continued until epithelialization is complete. In the case of invasive wound infection, systemic treatment with antibiotics along with topical treatment with ITF therapy can be administered.

### EXAMPLE 3

### Treatment of Herpetic Lesions

Patients suffering lesions caused by any of the herpes simplex viruses (HSV) can be treated with combination or monotherapy containing ITF. Herpetic lesions are typically on the face or genitalia and are treated with antiviral agents. Both HSV I and HSV II are transmitted by direct contact with an open lesion, or through secondary contact with infected objects. Thus, agents that promote epidermal healing will not only repair the cosmetic damage created by the lesion, it will also reduce the likelihood of viral transmission.

Presently, herpetic lesions are treated with standard antiviral therapy administered orally. According to this invention, ITF is administered concurrently in a topical preparation (e.g., paste or gel) at 5 mg/ml. Alternatively, the antiviral is coformulated with ITF for topical administration. For treatment of severe lesions, the amount of ITF can increased to 25 mg/ml, or more, and can be further combined with medications that relieve secondary symptoms. Corticosteroids, for example, may be included in the topical preparation, to relieve itching. Typically, the medicament is applied every 12 hours to the lesion until the outbreak subsides and the lesion is resolved. The lesion can be dressed with a bandage or gauze impregnated with the antiviral and ITF as an alternative, more convenient, means of drug delivery.

### EXAMPLE 4

### Treatment of Hand Dermatitis

Treatment of hand dermatitis is mostly concerned with avoidance of irritants, treatment of secondary infection, and reduction of inflammation. Lesions on the hand can be treated with cool dressings impregnated with trefoil peptides to dry and debride acute inflammatory lesions as well as to decrease swelling. Application of mid- to high potency topical glucocorticoid (e.g., 4% w/v hydrocortisone) formulated with 5 mg/ml ITF should also be administered to lesions. The topical steroid-containing trefoil peptides can be reapplied during each dressing change, or as often as required. The hands of an affected patient should be protected by gloves to keep the dressings, glucocorticoid, and ITF in place. If needed, a systemic steroid can also be administered. Treatment with topical antibiotics formulations containing trefoil peptides to limit secondary infections is also recommended.

What is claimed is:

1. A method for treating or preventing a lesion of the skin of a mammal comprising administering to the lesion, or the region of the skin where a lesion is to be prevented, a therapeutically effective amount of a trefoil peptide or biologically active fragment thereof.

2. The method of claim 1, wherein said trefoil peptide is spasmolytic polypeptide, pS2, or intestinal trefoil factor (ITF).

3. The method of claim 1, wherein said biologically active fragment is ITF₁₅₋₇₃, ITF₂₁₋₇₃, ITF₁₋₇₂, ITF₁₅₋₇₂, or ITF₂₁₋₇₂.

4. The method of claim 1, wherein said mammal is a human.

5. The method of claim 4, wherein said human is a preterm infant.

6. The method of claim 1, wherein said lesion is a traumatic lesion, a surgical lesion, a burn, or a pressure ulcer.

7. The method of claim 1, wherein said lesion is an allergic reaction, eczema, contact dermatitis, psoriasis, or acne.

8. The method of claim 1, wherein said lesion is caused by a bacterial, viral, or fungal infection.

9. The method of claim 8, wherein said lesion is caused by a herpes virus or a papilloma virus.

10. The method of claim 1, wherein said lesion is caused by antineoplastic therapy.

11. The method of claim 1, wherein said method further comprising administering to said mammal a second therapeutic agent or regimen.

12. The method of claim 11, wherein said second therapeutic agent is an ultraviolet blocking agent.

13. The method of claim 11, wherein said second therapeutic agent is an anti-inflammatory agent.

14. The method of claim 11, wherein said second therapeutic agent is a steroid.

15. The method of claim 14, wherein said steroid is a corticosteroid.

16. The method of claim 14, wherein said steroid is selected from the group consisting of fluocinolone, betamethasone, diflucortolone, fluticasone, mometasone, methylprednisolone, clobetasol, glucocorticoid, triamcinolone, hydrocortisone, fluticasone, budesonide, prednisone, prednisolone, methylprednisolone, dexamethasone, and beclomethasone.

17. The method of claim 11, wherein said second therapeutic agent is an antibacterial agent, an anti-fungal agent, or an anti-viral agent.

18. The method of claim 17, wherein said antibacterial agent is a penicillin, a cephalosporin, a tetracycline, an aminoglycoside, benzoyl peroxide, povidone iodine, azelaic acid, retinoid, clindamycin, or erythromycin.

19. The method of claim 17, wherein said anti-fungal agent is benzoic acid, undecylenic alkanolamide, ciclopirox olamine, polyenes, imidazole, allylamine, thiocarbamate, clindamycin, econaxole, fluconazole, flucytosine, griseofulvin, nystatin, clotrimazole, Amphotericin B, ketoconazole, enilconazole, itraconazole, butoconazole, tioconazole, or miconazole.

20. The method of claim 17, wherein said anti-viral agent is acyclovir.

21. The method of claim 11, wherein said second therapeutic agent is an analgesic agent.

22. The method of claim 21, wherein said analgesic is lidocaine, benzocaine, or an opiate.

23. The method of claim 11, wherein said second therapeutic is anthralin, a retinoid, a vitamin D analog, methotrexate, a benzodiazepine, or a cyclosporine.

24. The method of claim 11, wherein said trefoil peptide and said second therapeutic are administered in the same formulation.

25. The method of claim 11, wherein said trefoil peptide and said second therapeutic are administered in different formulations.

26. The method of claim 11, wherein said trefoil peptide and said second therapeutic are administered by different routes of administration.

27. The method of claim 11, wherein said trefoil peptide and said second therapeutic are administered within 24 hours of each other.

28. A pharmaceutical composition suitable for topical administration to the skin of a mammal, wherein said composition comprises a trefoil peptide or a biologically active fragment thereof and a pharmaceutically acceptable carrier.

29. The composition of claim 28, wherein said trefoil peptide or biologically active fragment thereof is spasmolytic polypeptide, pS2, or intestinal trefoil factor.

30. The composition of claim 28, wherein said biologically active fragment is ITF₁₅₋₇₃, ITF₂₁₋₇₃, ITF₁₋₇₂, ITF₁₅₋₇₂, or ITF₂₁₋₇₂.

31. The composition of claim 28, wherein said composition further comprises a mucoadhesive agent, or an osmotic agent.

32. The composition of claim 28, wherein said composition further comprises a second therapeutic agent.

33. The composition of claim 32, wherein said second therapeutic agent is an anti-inflammatory agent, or an analgesic.

34. The composition of claim 33, wherein said analgesic is lidocaine, benzocaine, or an opiate.

35. The composition of claim 32, wherein said second therapeutic agent is an antibacterial agent, an anti-fungal agent, or an anti-viral agent.

36. The composition of claim 35, wherein said antibacterial agent is a penicillin, a cephalosporin, a tetracycline, an aminoglycoside, benzoyl peroxide, azelaic acid, retinoids, povidone iodine, clindamycin, or erythromycin.

37. The composition of claim 35, wherein said anti-fungal agent is benzoic acid, undecylenic alkanolamide, ciclopirox olamine, polyenes, imidazole, allylamine, thiocarbamate, nystatin, clindamycin, econaxole, fluconazole, flucytosine, griseofulvin, clotrimazole, ketoconazole, enilconazole, itraconazole, butoconazole, tioconazole, miconazole, or Amphotericin B.

38. The composition of claim 35, wherein said anti-viral agent is acyclovir.

39. The composition of claim 32, wherein said second therapeutic agent is a steroid.

40. The composition of claim 39, wherein said steroid is a corticosteroid.

41. The composition of claim 39, wherein said steroid is fluocinolone, betamethasone, diflucortolone, fluticasone, mometasone, methylprednisolone, glucocorticoid, clobetasol, triamcinolone, hydrocortisone, fluticasone, prednisone, prednisolone, methylprednisolone, dexamethasone, beclomethasone, or budesonide.

42. The composition of claim 32, wherein second therapeutic agent is anthralin, a retinoid, a vitamin D analog, methotrexate, a benzodiazepine, or cyclosporine.

43. The pharmaceutical composition of claim 28, wherein said composition is a spray, ointment, paste, foam, lotion, gel, solution, or suspension.

44. A medical material comprising a trefoil peptide or a biologically active fragment thereof.

45. The medical material of claim 44, wherein said trefoil peptide is spasmolytic polypeptide, pS2, or intestinal trefoil factor.

46. The medical material of claim 44, wherein said biologically active fragment is ITF₁₅₋₇₃, ITF₂₁₋₇₃, ITF₁₋₇₂, ITF₁₅₋₇₂, or ITF₂₁₋₇₂.

47. The medical material of claim 44, wherein said medical material is selected from the group consisting of topical hydrogel dressings, patches, occlusive wound dressings, semi-occlusive wound dressings, tissue adhesives, sutures, and adhesive films.

48. The composition of claim 47, wherein said suture comprises material selected from the group consisting of gut, silk, collagen, glycolic acid polymer, and nylon.
